(19) United States
(12) Patent Application Publication
(10) Pub. No.: US 2016/0135715 A1
(43) Pub. Date: May 19, 2016

(54) METHOD FOR RESPIRATORY MEASUREMENT

(71) Applicant: TIDE MEDICAL OY, Tampere (FI)

(72) Inventors: Vile-Pekka Seppä, Tampere (FI); Jari Viik, Tampere (FI)

(21) Appl. No.: 14/902,357

(22) PCT Filed: Jul. 2, 2014
(86) PCT No.: PCT/FI2014/050547

§ 371 Date: Dec. 31, 2015

(30) Foreign Application Priority Data
Jul. 2, 2013 (FI) 20135726

(51) Int. Cl. A61B 5/08; A61B 5/087; A61B 5/091; A61B 5/00; A61B 5/053

(52) U.S. Cl. CPC A61B 5/0809; A61B 5/7203; A61B 5/053; A61B 5/091; A61B 5/6823; A61B 5/6824; A61B 5/087; A61B 5/7278

(57) ABSTRACT

The invention is directed method for measuring respiration using impedance pneumography over a duration of at least several minutes, several hours or over the duration of night sleep, the method comprising: using at least one electrode (11) configured to be in contact with an arm (2) of a human body (1) and at least one electrode (22) configured to be in skin contact with the thorax of a human body (1); defining impedance signal changes which relate to the respiratory volume changes or time-differentiated impedance signal changes which relate to the respiratory flow; and analysing variation over time in flow-time, volume-time, flow-volume curves, or derived numerical indices, or plain respiratory impedance signal or its time derivate over the duration of at least several minutes, several hours or over the duration of night sleep.

### DESCRIPTION OF THE RELATED ART

Diagnosis of asthma in preschool children is difficult because of unsuitability of the conventional lung function testing. However, measurements during spontaneous tidal breathing (TB) require minimal co-operation, thus being suitable for small children and infants. There is a large body of research suggesting that parameters derived from TB flow curves or flow-volume curves (TBFVC) change in a deterministic way with obstructive respiratory diseases in young patients. The studies have shown for instance that TB parameters relate to forced expiratory volume in 1 second (FEV1), airway resistance, bronchodilator response, and methacholine challenge and that they can be used to discriminate between pathological respiratory conditions. The current methods for assessing the TB pattern are hindered by the need of a direct access with the airways. Sedation can sometimes be used to overcome the psychological aspects of the measurement, but the physical face contact and the increased dead space still distort the respiratory pattern.

### SUMMARY OF THE INVENTION

The invention is directed to devices and methods for assessing a patient. The invention records respiration through thoracic impedance changes. The invention is preferably used for recording respiration over several hours or over the duration of night sleep preferably without calibrating the absolute respiratory impedance changes to absolute respiratory volume changes. The measured impedance is used to derive flow-time, volume-time, or flow-volume curves or numerical indices describing the curves. The curves, or the derived numerical indices, or plain respiratory impedance signal or its time derivate over a duration of at least several minutes, preferable several hours or over the duration of night sleep is analysed to gain information regarding the condition of the patient.

According to an aspect of the invention, there is provided a method for measuring respiration using impedance pneumography over a duration of at least several minutes, preferable several hours or over the duration of night sleep. The method comprises defining impedance signal changes which relate to the respiratory volume changes or time-differentiated impedance signal changes which relate to the respiratory flow preferably without calibrating the absolute impedance changes to absolute volume changes; and analysing variation over time in flow-time, volume-time, flow-volume curves, or derived numerical indices, or plain respiratory impedance signal or its time derivate over a duration of at least several minutes, preferable several hours or over the duration of night sleep.

According to an aspect of the invention, there is provided a method for measuring respiration using impedance pneumography over a duration of at least several minutes, several hours or over the duration of night sleep, the method comprising measuring a parameter for impedance pneumography by using at least one electrode configured to be in contact with an arm of a human body and at least one electrode configured to be in skin contact with the thorax of a human body, suppressing cardiogenic oscillations in a transthoracic electrical impedance signal, defining impedance signal changes which relate to the respiratory volume changes or time-differentiated impedance signal changes which relate to the respiratory flow, and analysing variation over time in flow-time, volume-time, flow-volume curves, or derived numerical indices, or plain respiratory impedance signal or its time derivate over the duration of at least several minutes, several hours or over the duration of night sleep.

In one embodiment the method further comprises suppressing the cardiogenic part of the measured impedance signal.

In one embodiment the method, further comprises deriving flow-time, volume-time, or flow-volume curves from individual or averaged multiple breaths from the impedance signal or the time-differentiated impedance signal.

In one embodiment the numerical indices describe the flow-time, volume-time, or flow-volume curves.

In one embodiment the indices relate to the shape of the curves, timing of peak values or other points of interest, or ratio of values or timings of two points of interest in the said curves.

In one embodiment the method further comprises analysing the curves, indices, or signals regarding their variation over time using methods such as nonlinear dynamics, entropy, detrended fluctuation analysis, Lyapunov exponent, correlation dimension, recurrence plot, noise limit, frequency spectrum or using descriptive statistics such as mean, variance, or distribution analysis.

According to an aspect of the invention, there is provided a method for measuring respiration using impedance pneumography over a duration of at least several minutes, preferable several hours or over the duration of night sleep preferably without calibrating the absolute respiratory impedance changes to absolute respiratory volume changes.

In one embodiment the method comprises suppression of the cardiogenic part of the measured impedance signal.

In one embodiment the method comprises defining impedance changes which relate to the respiratory volume changes or time-differentiated impedance changes which relates to the respiratory flow preferable without calibrating the absolute impedance changes to absolute volume changes.

In one embodiment the method comprises deriving flow-time, volume-time, or flow-volume curves from individual or averaged multiple breaths from the impedance signal or time-differentiated impedance signal.

In one embodiment the method comprises deriving numerical indices describing the said flow-time, volume-time, or flow-volume curves. Indices may be related to the shape of the curves, timing of peak values or other points of interest, or ratio of values or timings of two points of interest in the said curves.

In one embodiment the method comprises analysing the variation over time in the said flow-time, volume-time, flow-volume curves, or the derived numerical indices, or plain respiratory impedance signal or its time derivate over a duration of at least several minutes, preferable several hours or over the duration of night sleep.

In one embodiment the curves, indices, or signals regarding their variation over time are analyzed using methods such as nonlinear dynamics, entropy, detrended fluctuation analysis, Lyapunov exponent, correlation dimension, recurrence plot, noise limit, frequency spectrum or using descriptive statistics such as mean, variance, or distribution analysis.

One embodiment of the method uses two electrodes configured to be in contact with one arm of the human body and two electrodes configured to be in skin contact with the thorax of the human body on the side opposite to the arm. One embodiment of the method uses two electrodes configured to be in contact with opposite arms of the human body and two electrodes configured to be in skin contact with the thorax on opposite sides of the human body. The skin contact with the thorax is in one embodiment of the present invention the lateral thorax, on the side of the human body. In one embodiment the skin contact area with the thorax is the midaxillary line of the human body.

In one embodiment of the invention the skin contact between the arm and the torso is prevented by an insulation material configured to be positioned between the arm and the torso. The insulation material is made from a material known from its ability to insulate the electric current such as rubber or plastic. The material may be a hard object, such as a sheet of plastic positioned between the arm and the body, or it may have been formed to a shape to improve comfort. The insulation material may also be soft material; in one example the insulation material is configured to be a sleeve preventing the skin contact. The insulation material may also be configured to be a shirt or a vest preventing the skin contact.

Second aspect of the invention discloses an apparatus for measuring respiration using impedance pneumography over a duration of at least several minutes, several hours or over the duration of night sleep, the apparatus comprising: using at least one electrode configured to be in contact with an arm of a human body and at least one electrode configured to be in skin contact with the thorax of a human body; at least one processor and at least one memory including computer program code, the at least one memory and the computer program code arranged to, with the at least one processor, cause the apparatus at least to perform: defining impedance signal changes which relate to the respiratory volume changes or time-differentiated impedance signal changes which relate to the respiratory flow; and analysing variation over time in flow-time, volume-time, flow-volume curves, or derived numerical indices, or plain respiratory impedance signal or its time derivate over the duration of at least several minutes, several hours or over the duration of night sleep.

In one embodiment the invention comprises causing the apparatus at least to perform suppressing the cardiogenic part of the measured impedance signal. In one embodiment the invention comprises causing the apparatus at least to perform: deriving flow-time, volume-time, or flow-volume curves from individual or averaged multiple breaths from the impedance signal or the time-differentiated impedance signal.

In one embodiment the numerical indices describe the flow-time, volume-time, or flow-volume curves. In one embodiment the indices relate to the shape of the curves, timing of peak values or other points of interest, or ratio of values or timings of two points of interest in the said curves.

In one embodiment the invention comprises causing the apparatus at least to perform: analysing the curves, indices, or signals regarding their variation over time using methods such as nonlinear dynamics, entropy, detrended fluctuation analysis, Lyapunov exponent, correlation dimension, recurrence plot, noise limit, frequency spectrum or using descriptive statistics such as mean, variance, or distribution analysis.

In one embodiment the invention comprises two electrodes configured to be in contact with one arm of the human body and two electrodes configured to be in skin contact with the thorax of the human body on the side opposite to the arm. In one embodiment the invention comprises two electrodes configured to be in contact with opposite arms of the human body and two electrodes configured to be in skin contact with the thorax on opposite sides of the human body. In one embodiment the invention comprises preventing the skin contact between the arm and the torso by an insulation material configured to be positioned between the arm and the torso. In one embodiment the invention comprises the insulation material being configured to be a sleeve preventing the skin contact. In one embodiment the invention comprises the insulation material being configured to be a shirt or a vest preventing the skin contact.

The impedance pneumography (IP) overcomes the presented shortcomings of the conventional tidal breathing (TB) measurements. In addition, through continuous long-term TB pattern recording with ambulatory instrumentation, IP enables assessing the temporal manifestations of asthma that are receiving increasing research interest. It is a common misconception that the lung volume signal produced by the IP technique would stem solely, or at least mostly, from chest wall motion as in other noninvasive modes of respiration measurement. This would imply that abnormal respiratory mechanics could distort the IP measurement, and that IP would not be accurate enough to track subtle changes in the respiratory flow pattern. However, IP signal originates largely from the lung tissue, not only from the chest wall motion enabling making it potentially resistant to changes in breathing style. The presented study serves two purposes: Firstly, to show that abnormal respiratory physics, mechanics and control, as induced by intense expiratory loading, do not degrade the IP measurement accuracy, and secondly, to show that IP can be used to accurately reproduce the TBFVC and track its changes in individual subjects.

### DESCRIPTION OF THE FIGURES

The accompanying drawings, which are included to provide a further understanding of the invention and constitute a part of this specification, illustrate embodiments of the invention and together with the description help to explain the principles of the invention.

In the drawings:

FIG. 1 is a diagram illustrating the elements according to the invention,

FIG. 2 illustrates the sensor arrangement with a sleeve or a shirt,

FIG. 3 illustrates one embodiment of the sensor arrangement,

FIG. 4 illustrates the measurement setup used for simultaneous pneumotachograph and impedance pneumography tidal breathing recording. The current feeding electrodes of the impedance measurement (I+, I−) were placed on the fifth intercostal space on the midaxillary line and the voltage electrodes (V+, V−) on the arms in a matching position in the proximal side. The flow resistance was attached when indicated. An additional differential pressure sensor was attached to the mask to monitor mouth pressure for post-measurement detection of possible mask leaking,

FIG. 5 illustrates the difference between the normalized expiratory tidal breathing flow volume curves obtained with pneumotachograph and impedance pneumograph was calculated along each of the radial grey lines in steps of 10 degrees. The largest found difference, dₘₐₓ, was reported,

FIG. 6. illustrates expiratory tidal breathing flow-volume curves obtained simultaneously with impedance pneumography (black) and pneumotachograph (gray) during free (upper) and loaded (lower) breathing having largest difference d max between PNT and IP 5.6% and 5.0%, respectively,

FIG. 7 illustrates the largest difference (dₘₐₓ) between the normalized tidal breathing flow volume curves obtained with pneumotachograph and impedance pneumograph as illustrated in FIG. 4, wherein each dot represents one measurement and the lines denote the mean value.

FIG. 8 illustrates a table of exemplary measured parameters with free and loaded breathing,

FIG. 9 illustrates a tetrapolar bioimpedance measurement illustrating the paths of the current and voltage lead fields and their contribution to the negative, zero and positive measurement sensitivity areas, and

FIG. 10 illustrates definitions of various tidal breathing parameters.

### DETAILED DESCRIPTION

Reference will now be made in detail to the embodiments of the present invention, examples of which are illustrated in the accompanying drawings.

FIG. 1 is a block diagram illustrating the elements according to the invention. An apparatus for impedance pneumograhy 30 is connected via a connector interface 31 to the sensor 11 attached to the right arm 2 and the sensor 12 attached to the left arm 3 of a human body 1. Sensors 21, 23 are attached to the side of thorax or to the midaxillary line on both sides of the body 1. The sensor element comprises an electrode and a cable 13, 14, 15, 16 conducting the electrical signal to the connector interface 31. The midaxillary line is defined as a coronal line on the torso between the anterior axillary line and the posterior axillary line. The sensor placement may vary few centimetres from the midaxillary line.

Sensors 11, 12, 21, 22, cables, 13, 14, 15, 16, the interface 31 and the apparatus 30 are components of an impedance pneumography measurement system. The sensors 11, 12, 21, 22 may comprise a text, colour or other indication that helps the person using the impedance pneumography system to connect the sensor to a correct position on the body 1. Sleeves 41, 41 may comprise an indication separating the left arm 2 and the right arm 3. Also the sizing or the form of the sleeve 41, 42 may prevent the user from installing the sensor 11, 12 to a wrong position.

In one embodiment of the invention the interface 31 configured to the apparatus 30 is arranged to comprise indication of a correct installation procedure, such as colour coding or text. The apparatus 30 may also comprise a display for informing the user about the procedure. The software implemented in the apparatus 30 may also comprise code for providing assistive information to the user, confirming the correct installation procedure or informing about any errors during the installation or operation. One example of an error situation is the measurement data being out of the predefined range.

In one embodiment the computer program code comprises means for detecting the correct sensor 11, 12, 21, 22 being installed into the correct interface 31. The sensor may be configured to send information about the purpose or position in the interface 31 or the interface may have means for detecting the inserted sensor cable 13, 14, 15, 16.

The apparatus 30 may comprise an interface to transmit the impedance pneumography information to another device, such as a computer or another medical device. In one embodiment the apparatus 30 is arranged to convert changes in thoracic impedance resulting from respiration into a high level respiration signal that can be used with other applications. The apparatus 30 may also be integrated into another medical device.

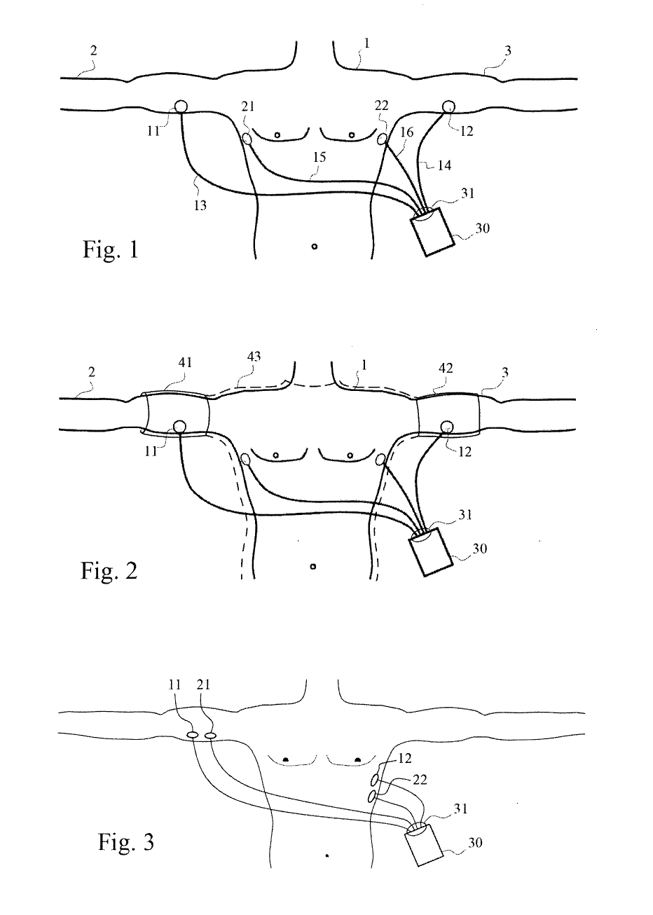

FIG. 2 illustrates one embodiment of the invention where sensors 11, 12 are arranged to be part of a sleeve 41, 42. The sleeve 41, 42 is made from electrically resistive material that prevents the direct skin contact between the arm 2, 3 and the torso. This prevents the electrical current from passing through the skin and thus contributing to false values. The bioimpedance values are measured through the high-axillary line or from the preferred path of the upper portion of lungs. The sleeve may also be part of a shirt or jacket 43 arranged to be used with the impedance pneumography system. The sleeve may also be in the form of an armband. In one embodiment the thickness of the armband keeps the arm at a distance from the body. The sleeve may also comprise the electrode configured as a fabric electrode made of suitable material such as silver or platinum.

Sensors 11, 12, 21, 22 may be arranged in different configurations. In a tetrapolar bioimpedance measurement four electrodes are used; two for feeding an alternating current of a constant amplitude and two for sensing the voltage. Also a constant voltage may be used while the current is measured. The electrode is measuring for example the voltage differential measured from both arms or the electrodes may be feeding the current to enable measuring of the impedance. The pair of electrodes purposed for the same parameter is always positioned to a distance from each other. For example electrodes feeding the current may be positioned to different arms. Alternatively one may be positioned to the arm and the other to the side of the thorax on the opposite side of the body, as illustrated in FIG. 3. Feeding the current and measuring the voltage may also be combined into a single sensor as a pair of electrodes.

In the impedance pneumography a small high frequency current is passed through a pair of skin electrodes and another pair of electrodes is used to record the generated voltage that is proportional to the impedance (Z), which again is proportional to the lung volume (V). The cardiogenic oscillations can be removed by a filtering technique described in the Finnish patent application FI20115110, which is incorporated by reference into this document.

Placing the electrodes 11, 12 on the arms 2, 3 improves significantly the linearity of the measurement results on a ΔZ/ΔV scale, especially at low lung volumes. One exemplary placement of the electrodes is between biceps and triceps brachii muscles. This placement of the electrodes on the arms can be described as placement on the supra-axillary line. Preventing the skin contact between the arms and the sides improves the measurement as the skin contact is not contributing to the bioimpedance value.

The improved linearity may result from the technical features and physiological features such as the motion and shape change of the thorax and thoracic organs, particularly the diaphragm and the liver, and small airway closing and alveolar collapse. After a deep exhale, and generally in lowered FRC, the diaphragm and liver reside more cephalad (headward) and, thus, are closer to the sensitivity field of the recording electrodes. This could be attributed to the finding that the ΔZ/ΔV nonlinearity occurs in deep exhales only in the infra-axillary electrode locations. Small airway closure and possibly alveolar collapse occur even in healthy young subjects when the lung volume is lowered below the FRC level. As the lung volume decreases, the smaller, intraparenchumal airways decrease in calibre until they close at low lung volume. During exhalation in an upright posture, this closure occurs predominantly and earlier in the lower part of the lung. As airway closure and alveolar collapse are known to affect the electrical impedance of the lung, this could partially explain why the infra-axillary configurations exhibit the presented ΔZ/ΔV nonlinearity.

Suppressing an oscillatory signal Sosc is carried out by providing a composite signal S comprising said oscillatory signal Sosc and a modulating signal Smod; high pass filtering the composite signal S with a high pass filter to produce an estimate of the oscillatory signal Ŝosc and an estimate of the modulating signal Ŝmod, wherein the estimate of the oscillatory signal Ŝosc comprises first oscillations during a first state of the modulating signal Smod and second oscillations during a second state of the modulating signal Smod; defining a first bin associated with said first state and a second bin associated with said second state; assigning the first bin for said first oscillation according to a state defined from the estimate of the modulating signal Ŝmod and the second bin for said second oscillation according to a state defined from the estimate of the modulating signal Ŝmod; forming a first average waveform for said first oscillations in said first bin and a second average waveform for said second oscillations in said second bin; and using said first and second average waveforms for suppressing said oscillatory signal Sosc from said composite signal S in the respective states of said first and second average waveforms.

In other words, an oscillatory signal Sosc can be suppressed from a composite signal S comprising the oscillatory signal Sosc and a modulating signal Smod without removing parts of the modulating signal Smod. The composite signal S is high pass filtered to produce estimates of oscillatory signal Sosc and the modulating signal Smod. The estimate of the oscillatory signal Ŝosc comprises at least first oscillations during a first state of the modulating signal Smod and second oscillations during a second state of the modulating signal Smod. A first bin associated with said first state and a second bin associated with said second state are defined and the first bin for said first oscillation according to a state defined from the estimate of the modulating signal Ŝmod and the second bin for said second oscillation according to a state defined from the estimate of the modulating signal Smod are assigned. A first average waveform for said first oscillations in said first bin and a second average waveform for said second oscillations in said second bin are formed. And these first and second average waveforms are subtracted from the composite signal S in the respective states of said first and second average waveforms to form the modulating signal Smod. The method may be applied, for example, for suppressing the cardiogenic oscillations in an impedance pneumography signal, wherein the cardiogenic oscillations and the impedance respiratory signal form a transthoracic impedance signal.

In the following exemplary means; for example, mask, pneumotachograph, calibration syringe, pressure sensor and resistor element; are disclosed in connection with the invention. These means may be used to verify the accuracy of the results, and thus may not be needed by the invention itself.

In the following the testing method of the implementation according to the invention is explained.

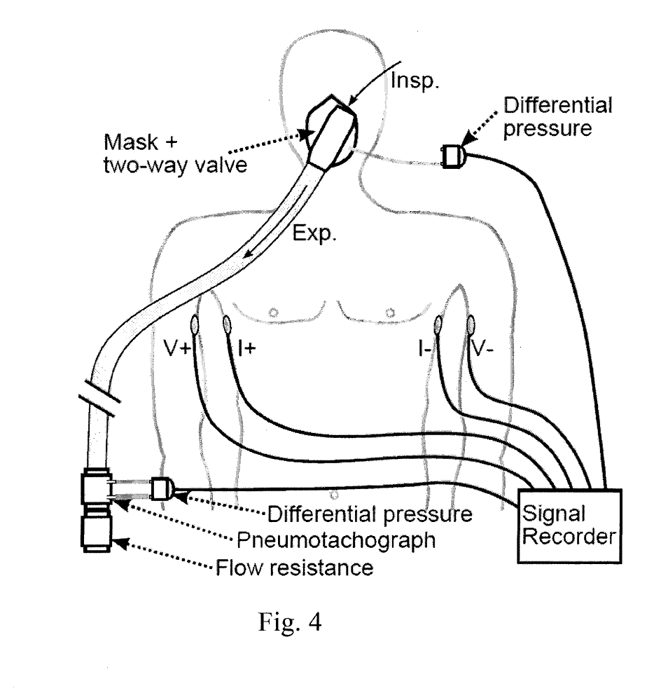

Subjects and Procedures: The subjects were 17 healthy young subjects (age 22-28, body mass index 19.2-26.9, 4 females) with no self-reported chronic respiratory diseases. The study was approved by the institutional review board and a written consent was obtained from all participants. Three minute recordings of tidal breathing were acquired simultaneously with a pneumotachograph attached to the expiratory limb of the system and with an IP system, FIG. 4. The measurements were conducted in supine position and the recording was repeated after attaching a flow resistor element on the expiratory limb. The current feeding IP electrodes were placed on the fifth intercostal space on the midaxillary line and the voltage measurement electrodes on the same level on the proximal side of the arm between the biceps and triceps brachii muscles. This electrode configuration has been previously reported to produce a highly linear impedance change to lung volume change ratio. In addition, single channel ECG was measured to enable the use of a signal filtering algorithm that removes the cardiogenic part of the impedance signal.

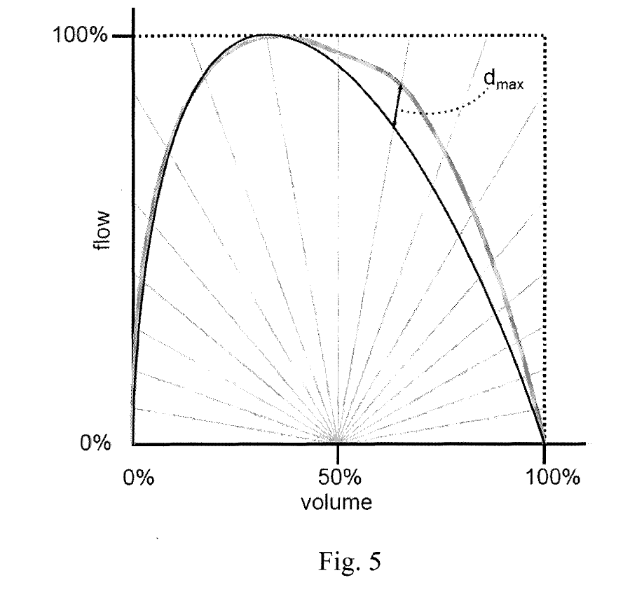

Equipment: The subjects wore a face mask (7900 Series, Hans Rudolph, Shawnee, Kans. 66227, USA) of the best fitting size (XS, S, M, L). The mask was connected to a two-way valve system (Series 2700 Large, Hans Rudolph) where the inspiratory limb was free and the expiratory limb was connected to the PNT and the 15 cm H 2 O s/l resistor element (7100R, Hans Rudolph) with a three meter tube. For a post-measurement detection of mask leaks, the mouth pressure was continuously recorded inside the mask with a pressure sensor (SS42L, Biopac Systems). The heated PNT (A. Fleisch No. 3, Lausanne, Switzerland) was connected to a differential pressure transducer (SS40L, Biopac Systems, Goleta, Calif. 93117, USA) with a declared combined linearity and hysteresis error of ±0.05%. The flow measurement system was calibrated before each subject using a three-litre calibration syringe (PCS-3000, Piston Medical, Budapest, Hungary). IP signal was recorded with a bioimpedance measurement device (EBI100C, Biopac Systems) using a 100 kHz, 400 μA excitation current. All the transducers were connected to a Biopac MP35 unit that digitized and stored the signals at 500 Hz sampling frequency. 3) Signal Processing and Statistical Analysis: In addition to the respiratory component, the thoracic impedance signal also contains a cardiogenic component that originates from the pulsatile blood movement in the thorax. This distortive part of the signal was attenuated using the filtering algorithm. For producing the TBFVCs a number of breaths were averaged. The most similar respiratory cycles were discovered from the IP signal by an algorithm based on comparing the correlations of the flow signals of the cycles. If less than four similar cycles were found due to slow and irregular breathing, the measurement was excluded from the analysis. Each TBFVC was normalized in volume and flow for range 0 . . . 100% and the chosen individual TBFVCs were averaged in 100 angle segments in a manner resembling the one illustrated in FIG. 5 to produce a single representative TBFVC. Then the corresponding cycles in the PNT recording were normalized and averaged in the same way. The difference between IP and PNT for each pair of averaged TBFVCs was assessed by calculating their difference along radial lines with 10 degree separation and choosing the highest of those values to represent the difference d max as illustrated in FIG. 5. The statistical difference between measurements during the free and loaded breathing was assessed by the paired Wilcoxon signed rank test.

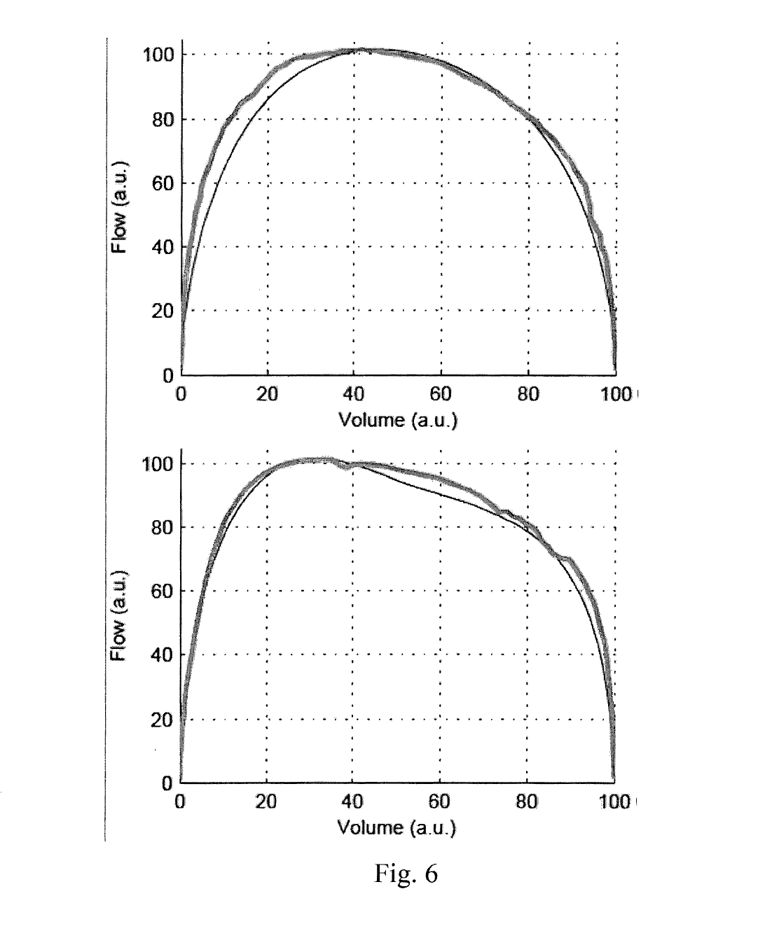

Results: Two successful recordings of TBFVC were obtained from all but one subjects simultaneously with PNT and IP yielding a total of 33 measurements. For one subject the loaded respiration was too irregular to produce four similar TBFVCs as required for the analysis. The amount of similar breaths included in the averaging of the TBFVC ranged from 6 to 57 (mean 28.1). The expiratory loading produced strong changes in the TBFVCs, FIG. 6, along with significant (p<0.05) changes in peak expiratory mouth pressure (PEPm), tidal peak expiratory flow (TPEF), expiratory time (tE), ratio of inspiratory to expiratory time (tI:tE), and respiratory rate (RR) as measured by the PNT in FIG. 8.

Peak expiratory mouth pressure (PEPm), tidal peak expiratory flow (tpef), expiratory time (tE), ratio of inspiratory to expiratory time (tI:tE), respiratory rate (RR), and tidal volume (VT) given as mean±SD obtained with a pneumotachograph illustrate the effect of the expiratory loading on respiration. *: p-value<0.05.

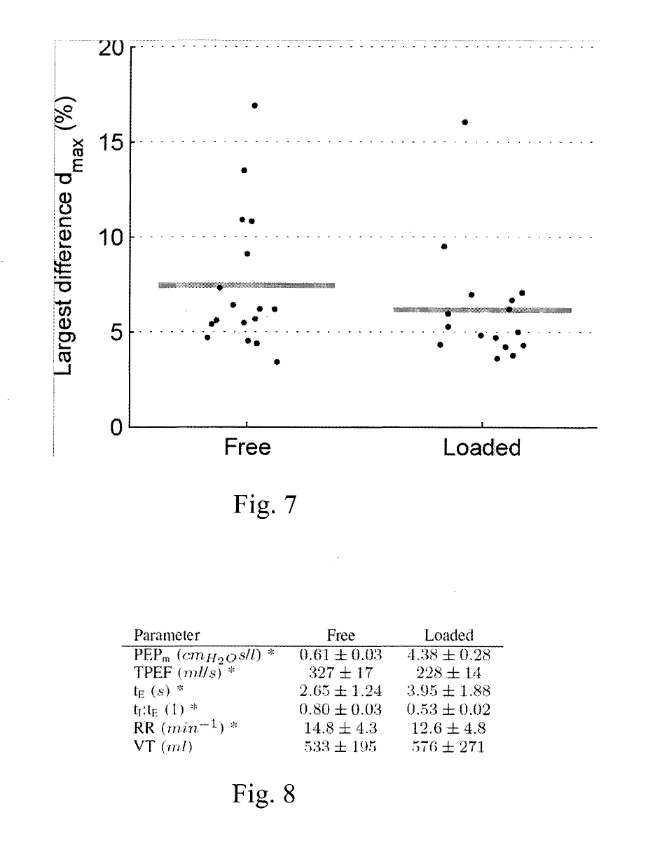

The difference between the IP and PNT TBFVCs as assessed by the dₘₐₓ value was found to be 7.2%±3.1% and 7.3%±4.2% during free and loaded breathing, respectively in FIG. 7. Most measurements (28 of 33) were found to have d max below 10%. The difference in dₘₐₓ values between free and loaded breathing was not statistically significant (p=0.46).

The intense expiratory loading used produced clear effects on the control and mechanics of the breathing (Table I). This poses multiple potential hazards for the accuracy of IP measurements for example through substantial changes in the cyclic pattern of the respiratory muscle activation and motion of the chest wall, and changes in cardiorespiratory coupling and cardiac mechanics. Indeed, one of the major difficulties in accurate respiratory flow measurement with IP has been posed by the cardiogenic oscillations (CGO). Recently a filter algorithm based on CGO ensemble averages modulated by instantaneous lung volume was proposed to solve the problem. This brings further evidence of its efficacy under conditions that are rather complex from a cardiopulmonary standpoint. Namely, the pleural pressure changes modulate the heart rate and stroke volume [21], which contribute to the shape and frequency of the measured CGOs. Furthermore, the electrode placement is most important in determining the dynamic ratio between the lung volume changes and the measured impedance. The electrode configuration used in this study had been previously presented only for prone subjects, but was now used in the supine position and found to work appropriately. We conclude that the agreement between normalized TBFVCs produced with PNT and IP was found excellent. This was enabled by correct electrode positioning and appropriate filtering of the cardiac impedance signal. The agreement was not affected by change in respiratory flow pattern or the changes in the respiratory mechanics as induced by the respiratory loading.

Narrowing of the lower airways of the respiratory system are a common source of shortness of breath and a typical feature of diseases such as chronic obstructive pulmonary disease (COPD) and asthma. Conventionally the narrowing, or obstruction, is assessed in a lung function laboratory with a spirometer. In spirometry the subject conducts a forced exhale manoeuvre and the resulting air flow at mouth is measured. There are, however, patient groups such as the elderly, disabled, intensive care patients, and young children and infants, who cannot adequately perform the required manoeuvres. For instance, the diagnosis of childhood asthma is often qualitative, time-consuming and difficult due to the limited methods for the paediatric lung function assessment. However, as is intuitive, the air flow limitation in the lungs manifestates also in the tidal breathing (TB) air flow profile, not only in the forced breathing. Measurement of TB is suitable for practically any patient regardless of age or condition. Considerable effort has been put forward to find indices from the tidal air flow that would reliably tell about the presence or severity of airway obstruction. This has proven difficult, due to the multitude of mechanical, neurological, physiological, psychological and instrumentation-related factors that affect TB. Especially the conventional measurement equipment using a mouth piece of a face mask has been shown to alter the respiratory control through increased dead space and facial nerve stimulus. These problems and the cognitive factors could be removed if TB was measured at normal living conditions with noninvasive methods. Moreover, noninvasive equipment could allow TB measurement over extended periods of time, revealing the spontaneously occurring nocturnal obstruction of asthma and enable analysing the fractal and chaotic features of respiration in a natural setting. There is a wide selection of noninvasive respiration measurement instruments for research and clinical purposes. However, the techniques have been used for, and are usually capable only of, monitoring the respiratory rate (RR) or tidal volume instead of the respiratory air flow profile. This deficit in accuracy may be attributed to the measurement principle of most methods, which is based on assessing the movement of the chest wall. One of the earliest methods, impedance pneumography (IP), is however based on measuring the respiration-induced changes of electrical conductivity (or impedance) within the chest using electrodes placed on the skin surface. One of the earliest investigators to describe the phenomenon of respiration-induced impedance changes were Atzler & Lehmann (1932). They were analysing the impedance variations of cardiac origin, cardiogenic oscillation (CGO), and discovered that respiration distorted their measurements. Nearly three decades later, the first paper to quantitatively describe the respiratory variation, IP, was published with two key concepts that have remained in the centre of the IP research. Firstly, the need to obtain a linear relationship between the lung volume and the measured impedance, and secondly, the need to attenuate the CGO. Linearity between lung volume and impedance depends heavily on placement of the skin electrodes. The general agreement has been that when the electrodes are placed on the sides of the thorax, a higher placement closer to the axilla yields amore linear relationship. There is evidence that this is because at higher location the impedance variations stem from variation in the lung tissue aeration, as desired, whereas in the lower locations other factors such as the movement of the diaphragm contribute to the signal. Attenuation of the CGO needs to be done efficiently, but without distorting the respiratory part of the impedance signal. This is difficult due to several features of the signals, most prominently their nonstationarity and overlap in the frequency spectrum. Several signal processing methods to decompose the respiratory and cardiac part have been proposed, but thus far the results have been either unsatisfactory or lack proper clinical validation.

Electrical impedance refers to the properties of a physical object that oppose (impede) the flow of electrical current through it. Bioimpedance refers to the electrical impedance of biological matter, such as living human tissues. The measurement of Bioimpedance of human tissues, organs or body parts gives information on their state or function. Bioimpedance measurement has received wide and long-lasting research interest because of its noninvasiveness and apparent simplicity. Generally the bioimpedance measurement is based on the Ohm's law relating the imaginary quantities of impedance Z, current I and voltage U as Z=U/I. In practice, an alternating current is generated and led through the tissues by the measurement instrument while measuring the voltage generated by the current. The measurement instrument is connected to the tissues by electrodes, which are essentially transducers transforming the electron-carried current in the cables and electronics into an ion-carried one in the biological substance, and vice versa. Most measurement solutions feature two or four electrodes and are referred to as bipolar or tetrapolar, respectively. Regarding the current feeding, the most obvious and most used approach is to construct an electric circuit, a current source that aims to provide as stable as possible current into the tissue. An example of such circuitry is the Howland current pump. However, depending on the application, the requirements for the stability of the current source under varying loads (impedances) may be rather strict and difficult to full. Thus, an approach where the current source is actually a simpler voltage source and the generated current is measured, is gaining more foothold. Either way the basic principle of Z=U/I prevails. The bioimpedance measurements can be divided into categories of frequency domain and time domain assessments. In the first one the current is fed at several different frequencies, often referred to as impedance spectroscopy. This can be realised by applying consecutively sinusoidal currents of different frequency or by feeding a composite signal consisting of several frequencies that is then decomposed by means of signal processing. In the bioimpedance context, impedance spectroscopy has been used in a variety of in vivo and in vitro applications such as stem cell growth, patient hydration status and body composition, biopsy needle guiding through tissue type recognition, and wound healing monitoring. In the time domain impedance is assessed at a single frequency but continuously over time in order to capture temporal impedance variations stemming from physiological functions. Modern instrumentation allows combining both domains and also conducting multiple non-interfering bioimpedance measurements simultaneously in the same body but their applications are still very few. Most of the efforts in the time domain have been directed towards ICG where haemodynamic parameters such as stroke volume are determined from thoracic impedance signals. The other main application of time domain impedance measurement is in assessing the respiration, impedance pneumography. Defining the components that contribute to a measured impedance in an electronic circuit is rather unambiguous whereas in a homogeneous biological volume conductor it is not. One cannot model the thorax as a series or parallel connected circuit consisting of the lungs, heart, bones etc. Instead, in a volume conductor the current forms distribution, a vector field, that is called the lead field. The current forms a spatial distribution that avoids any regions of higher impedance and favours the ones of lower impedance. The same applies for the lead field of the voltage measurement according to the principle of reciprocity. In fact, interchanging the current and the voltage lead fields has no effect on the measured impedance, apart from the effects that stem from non-idealities in the measurement instruments. The current lead field JLI and the voltage lead field JLE combined form the sensitivity field S of the impedance measurement S=J_{LE}·J_{LI}. The measured impedance of a volume conductor V is thus obtained by integrating the inverse of conductivity and the product of the sensitivity field at each point within the volume as

z=∫_{V}1/σS=∫_{V}1/σJ_{LE}·J_{LI}.

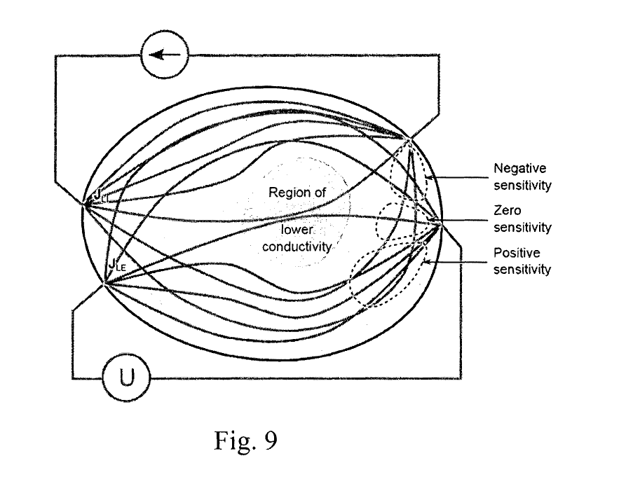

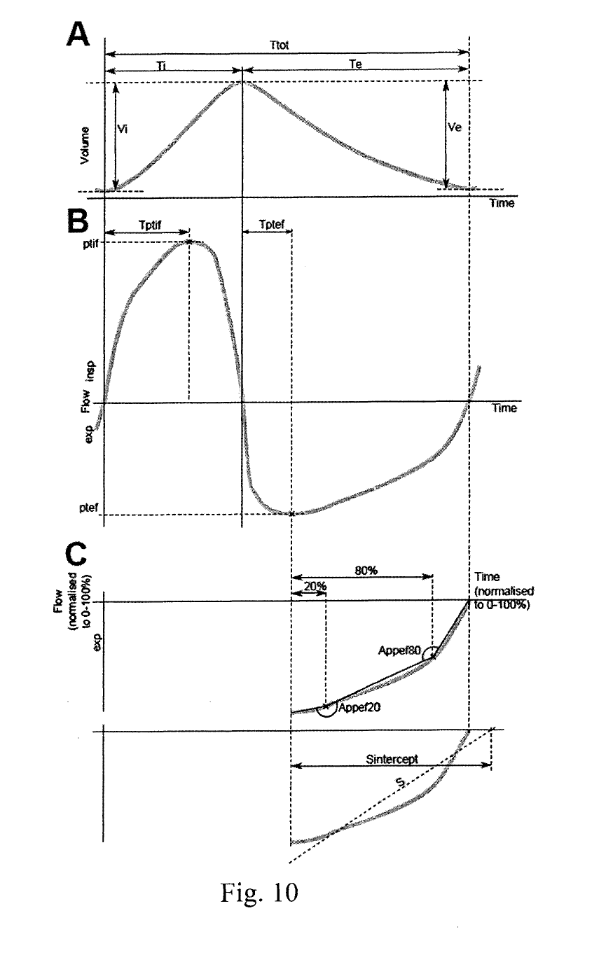

There are important aspects to consider in how the lead fields interact to form the sensitivity field as their dot product. In a bipolar measurement, the current and the voltage are applied through the same pair of electrodes which implies that their leadless are uniform and the sensitivity field (dot product of the lead fields) is always positive. In a tetrapolar setting the situation is not as straightforward. As illustrated in FIG. 9 the sensitivity field may form areas of zero sensitivity and even negative sensitivity. In the areas of negative sensitivity an increase in impedance contributes as a decrease in the total measured impedance. In areas where the fields are perpendicular or only either field is present, the sensitivity is zero. However, this complexity of the tetrapolar measurement brings important advantages over the bipolar one. Firstly, it allows focusing the sensitivity field spatially in a more elegant way when used correctly. For instance one may exclude the tissues close to the surface of the body. Secondly, perhaps as more widely recognised feature, because the two lead fields do not meet in the connectors, cables, electrodes or electrode-tissue interfaces, these unwanted distortive components of the system will not contribute to the measured impedance.

The thoracic impedance signal consists of a cardiac and a respiratory component. For acknowledging the benefits and limitations of IP, it is important to understand what creates the measured respiratory impedance signal. General conclusions from the literature on the source of respiratory impedance signal are that 1) the lung tissue itself has linear volume-impedance relationship, and that 2) the thoracic respiratory impedance changes can be seen with both low and high bilateral midaxillary electrode placements, but the low ones may reflect other respiratory sources than lung tissue aeration and are thus less linear with lung volume changes than the high ones.

Electrode locations. The location of the electrodes on the body affects the magnitude of the respiratory, cardiac, and motion artefact signals and importantly, the linearity of the Z/V ratio. Depending on the application, different features are favoured. For instance, in ICG, one naturally would prefer a strong cardiac component and negligible respiratory component, whereas for IP the preference is obviously opposite. Usually most important feature is linearity with the physiological phenomenon of interest. In IP this means linearity with lung volume changes. Electrode placement is the main determinant of this linearity. The present invention utilizes electrode placements regarding their linearity during more demanding respiratory manoeuvres. A high midaxillary electrode placing is not very linear when the test manoeuvre includes also deep exhales. Instead, in a tetrapolar setting, placing the other electrode pair high on the midaxillary line and the other electrode pair in the arms, opposing the first pair, yields a highly linear Z/V at all lung volumes during a VCM.

An obvious aim for IP research has been to obtain absolute ventilation volume values such as tidal volume and minute ventilation in a noninvasive and even ambulatory manner. As the Z/V ratio is known to have a high intersubject variation, there are two potential ways to achieve absolute measures. Firstly, to find a regression equation that uses a priori information such as body geometry and tissue conductivities to convert impedance changes to volume changes, or secondly, to calibrate impedance measurement by breathing at a known volume. The first approach would often be more beneficial, but has proven difficult to establish. The challenges include at least variation in human anatomy, and difficulty in determining the measurement sensitivity field and tissue conductivities. Prior art discloses a Z/V equation which included tissue conductivities, electrode distance (red) and the measured base resistance. Another known very simple equation is log(Z/V)=2.656−1.08 log(W) where W was the body mass in kilograms. The second approach for obtaining the Z/V ratio would be to simply measure the respired volume during a calibration period. For subjects in a static position such as patients in an intensive care unit (ICU) this could be feasible at least for limited time periods, but in mobile subjects the postural change causes changes in the Z/V ratio, often in a rather unpredictable manner. Even in the ICU patients the Z/V may likely change due to for instance edemic fluid accumulation. The solutions according to prior art indicate too many factors that affect the absolute impedance readings to establish a reliable Z/V equation and that IP has clinical value only in monitoring of respiratory rate and apnoeas.

Thoracic impedance recording is susceptible to motion artefacts created by for example walking or arm movement. The artefacts can be rather strong, masking the signal of interest. In certain applications, such as apnoea monitoring, it would be important to remove the artefact or at least reliably detect between the artefact and the physiological event of interest.

The cyclic pumping action of the heart muscle moves blood within the thorax. This creates a pulsatile measurable impedance signal. This signal can be either desired as in ICG or distortive as in IP. In the following the cardiac impedance signal is discussed from the IP standpoint. Although cardiogenic impedance variations have been studied thoroughly by the investigators of ICG, they have received little attention from the investigators of IP. Unfortunately the electrode placements that have been used in ICG studies are fundamentally different from the ones used in IP studies. This greatly limits the usefulness of the ICG studies in understanding the cardiogenic impedance signal present in the IP. One reason for the IP investigators to put so little effort in understanding the CGO could be that firstly, obviously, IP focuses on the lungs, not the heart, but secondly, the applications of IP have been thus far rather unambitious, permitting the use of heavily distortive filtering methods in the removal of the CGO or even simply ignoring atone of the main questions in understanding the CGO within the IP context is what is the relative contribution of the systemic and pulmonary circulation to the signal. This can be studied by injecting highly conductive saline solutions to the circulatory system at different locations and observing the resulting impedance changes.

Attenuating the cardiogenic oscillations. The effective attenuation of the cardiogenic impedance part is essential if respiratory variables more sophisticated than tidal volume or respiratory rate are to be derived from the signal. Furthermore, it is highly important that the method of attenuation does not deteriorate the respiratory part of the impedance signal. The problem is not a trivial one mainly for two reasons: 1) Even though the main power band of the respiratory signal component is clearly at a lower frequency than the cardiac signal, the respiratory signal contains harmonics that overlap with the cardiac signal frequency. 2) Neither of the signal components are stationary, meaning they change over time. Despite the variety of methods proposed for solving the problem, most prior art solutions lack objective clinical evidence regarding their performance, or they are assessed only from the cardiology standpoint, leaving their applicability to IP unknown. It is clear from the medical literature that the cardiac and respiratory impedance signals are non-stationary and mutually dependent. Nor can it be assumed that the impedance measurement sensitivity distribution would not change with respiration. Moreover, the cardiac or respiratory part of the impedance signal measured simultaneously at two distinct locations of the thorax or the body are not simply linear combinations of each other. Instead, there is likely time delay and waveform shape difference between the two simultaneous measurements at distinct body regions.

Conventional lung function assessment methods, spirometry and peak expiratory flow (PEF) meter, require that the subject performs demanding respiratory manoeuvres in repeatable manner. This hinders or prevents their use in patients with limited cooperation such as infants, preschool children, elderly or disabled people and subjects for whom deep inspiration causes pain, such as thoracic surgery patients. Nevertheless, measurements of restful spontaneous TB are feasible even in such patient groups. Close analysis of respiratory flow within each breath and from breath-to-breath yields useful information on the lung health condition of the subject, especially in the presence of airway obstruction.

Quantification of tidal breathing. Although the early observations on clinical relevance of TB analysis were rather descriptive or subjective, the need for numerical indices to describe TB was soon realised. Several quantification methods and their clinical relevance have been described. Most of the work has been directed towards simple indices that relate to the shape of the tidal flow curve much like in conventional forced spirometry. Another analysis approach is based on assessing the time dynamics or complexity of respiratory flow signals or other lung function measurements over time.

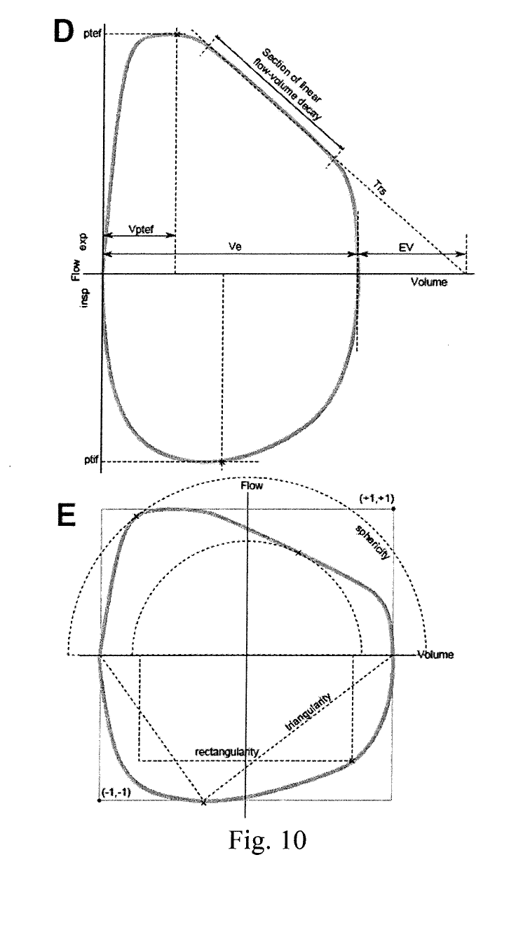

Analysis of the tidal flow curve shape. A list covering most of the TB parameters that have been studied in a clinical settings are presented in Table A with the clarifying illustrations in FIG. 10.

TABLE A
Tidal breathing parameters

| Name/ | | |
|---|---|---|
| Abbreviation | Unit | Description |
| Tptef/Te | 1 | Ratio of time to reach tidal PEF to |
| | | total expiration time |
| Tptif/Ti | 1 | Ratio of time to reach tidal PEF to |
| | | total inspiration time |
| Vptef/Ve | 1 | Ratio of volume at tpef to total |
| | | expired volume |
| ptef | L/s | Peak expiratory tidal flow |
| S | 1 | Slope of a line fitted to the post- |
| | | peak part of the expiratory flow-time |
| | | curve |
| EV | L | Extrapolated volume that is obtained |
| | | by fitting a line in the linear |
| | | portion of the expiratory FVC (Note: |
| | | no unambiguous mathematical |
| | | definition) |
| Trs | s | Time constant of the respiratory |
| | | system. The slope of a line fitted to |
| | | the linear portion of the expiratory |
| | | FV curve defined as Trs = tan(a). |
| Dtr/Te | 1 | Ratio of time from beginning of |
| | | exhale until beginning of the linear |
| | | part of the FV curve to total |
| | | expiratory time |
| \flow b-o | L/s | Expiratory flow at the point of end- |
| | | expiratory flow break-off (premature |
| | | onset of inspiration). |
| ptef/Tptef | L/s2 | Mean initial expiratory gas |
| | | acceleration |
| (ptif + ptef)/Vt | 1/s | Axis ratio of the TBFVL |
| Sphericity, | 1 | Parameters that describe the shape of |
| triangularity, | | expiratory or inspiratory FVL as to |
| rectangularity | | how closely it resembles a sphere a |
| | | triangle or a rectangle. |
| Polynomial fit | 1 | The coefficients of a first or second |
| coefficients | | order equation fitted on the |
| | | normalized expiratory or inspiratory |
| | | FVC |
| tef50 | L/s | Tidal expiratory flow at 50\% of |
| | | total exhaled volume. Similarly for |
| | | other percentages or inspiratory |
| | | flows (tif) |
| tef25/ptef; | 1 | Ratio of tidal flow at 25\% or 50\% |
| tef50/ptef | | of total exhaled volume to ptef |
| Ti/Te | 1 | Ratio of duration of inspiration to |
| and | | duration of expiration or vice versa |
| Te/Ti | | |
| Ti/Ttot | 1 | Ratio of duration of inspiration to |
| | | duration of complete respiratory |
| | | cycle |
| Vt/Ti | L/s | Mean inspiratory flow |
| s | 1 | Exponential of the power law |
| kd | 1 | Harmonic distortion in the frequency |
| | | spectrum of the respiratory flow |
| | | signal |
| Appef20 | | Angle of a regression line passing |
| | | through four points in the post PEF- |
| | | time profile at point of 20\% of |
| | | expiratory time |
| Appef80 | | Angle of a regression line passing |
| | | through four points in the post PEF- |
| | | time profile at point of 80\% of |
| | | expiratory time |
| Ippef | | Integral of post-PEF-time profile |
| Vt | L | Tidal volume |
| RR | 1/min | Respiratory rate |
| MV | L/min | Minute ventilation |

Analysis of the tidal flow dynamics. Consider some imaginary TB parameter assessed at nine moments in time. For patients A and B this series would yield values of 1, 3, 3, 1, 2, 1, 3, 2, 2 and 1, 2, 3, 1, 2, 3, 1, 2, 3, respectively. Assessed by conventional descriptive statistics, these two series both have mean of 2.00 and variance of 0.75, yet it can be seen that the series of patient B is obviously periodic whereas that of patient A is not. Differentiating between these two patients requires analysing the time dynamics of the series. The time dynamics analysis includes several overlapping concepts such as entropy, chaos, fluctuations, fractality, nonlinearity, and complexity. Techniques based on these concepts have been applied widely in the analysis of physiological phenomena, but have yet to reach clinical approval in most cases. In pulmonology the time dynamics analysis is applicable at least in the measurements of PEF, airway mechanical impedance and tidal air flow. Most of the time dynamics are cross-sectional or interventional (typically bronchodilation (BD) or MIB), but there are some longitudinal ones as well and, interestingly, some prospective ones. For instance, the presence of long-range correlations (detrended fluctuation analysis) in the PEF time series is associated with a decreased risk of asthma exacerbation within one month, regardless of the present PEF value. Indeed, it has been postulated that the analysis of time dynamics of pulmonary measurements could yield information on presence of an underlying respiratory disease, and of its severity and course, regardless of its current clinical symptom presence. At a very general level, it can be said living organisms require a delicate balance between stability (order) and variability (chaos) in their physiological functions. Diseases are characterised by either overly stable or overly variable functions. Physiological relevance of the tidal flow curve shape. During spontaneous respiration the neural feedback system controls the muscles to overcome the mechanical impedance of the airways and to achieve ventilation at the alveolar level. An increase in the mechanical impedance, such as bronchial obstruction, will affect the neural control which again, should be witnessed as a change in the TB flow pattern. This leads to a characteristic response in respiration. However, the TB parameters are not direct surrogates of measures of mechanical impedance of the airways. The measurable TB flow signal is a synthesis of multiple interacting factors such as the passive mechanical impedance, respiratory neural control, and glottic aperture (one could complement the list with effects of measurement equipment and cognitive state of the subject at least). Despite the subtle nature of the TB analysis, as more sophisticated signal processing methods are applied, TB may reveal itself to be useful in the diagnosis of pulmonary diseases. In the following the relevance of selected TB parameters is discussed from the clinical and respiratory mechanics standpoint. Tptef/Te and the highly correlated Vptef/Ve have been studied extensively with respect to their physiological origin and their clinical meaning. From the physiology standpoint it has been established that expiratory braking is an important determinant of the Tptef/Te. In normal subjects the activation of the inspiratory muscles continues well into the expiration. According to an example, the mean time for muscle activity to reduce to 50% and 0% amounted, respectively, 23% and 79% of the expiratory time. In another example Tptef/Te can be controlled by inhibiting or exciting the inspiratory muscles during expiration. The expiratory braking diminishes in the presence of airway obstruction. These findings are considered to explain why Tptef/Te is reduced with obstruction. The Tptef/Te and Vptef/Ve only involve the beginning of the expiration. This may be shown by presenting the parameters of respiratory time constant (Trs) and extrapolated volume (EV). According to the basic model of the lung mechanics having constant compliance and resistance, in a relaxed expiration there is a linear flow-volume relationship. This slope is Trs and by analogy with an electrical model it is equal to resistance time's compliance of the total respiratory system. Thus, if they would be able to correctly identify the section of relaxed expiration (linear decay in flow-volume plot) that part should give information about severity of airway obstruction. Indeed, in their study on 112 adult patients they received correlations between EV and FRC % pred (r=0.68, p<0.001), and Trs and airway resistance (r=0.65, p<0.001). However, a limitation of this method is the ambiguous manual identification of the linear part of the expiratory FVC. In patients with airway obstruction the whole expiration maybe a passive, relaxed event, but in healthy subjects the inspiratory neuronal drive continues in the exhalation. One study circumvented the manual identification by setting a fixed region between 60% and 90% of expired volume on which the Trs line was fitted. They excluded breaths where the correlation coefficient between the line and the flow-volume-loop section was below 0.80 or if Tptef/Te was above 40%. The parameter S and its intercept with the zero flow rely on similar physiological rationale as Trs and EV. The major difference between S and Trs is that S is defined in the flow-time curve whereas Trs is defined in the FVC. In addition, S is defined mathematically unambiguously whereas Trs needs manual identification of the linear portion of the FVC.

Technical aspects of tidal breathing measurement, conventional measurement equipment. The European Respiratory Society (ERS)/American Thoracic Society (ATS) task force on infant TB analysis states that the standard equipment for clinical TB measurement is a face mask connected to a PNT. It is however necessary to be aware of the effects that the most commonly used face mask and PNT equipment may have on the results. One study compared VT and Tptef/Te values and their variation between immediate placing of the face mask and few minutes later. They found that VT was initially significantly smaller and Tptef/Te more variable as compared to the later moment. Another study corroborated this finding with same equipment, but found that this effect was not seen with flow-through technique (FTT) equipment that does not increase the respiratory dead space. A similar finding with FTT was made, but they noticed that even with FTT the Tptef/Te values were less variable few minutes later than immediately after placing the mask. One discovery is that the placement of the mere rim of a face mask without any dead space addition affects significantly affects the TB pattern, supposedly through trigeminal nerve stimulation. These effects could be entirely removed by noninvasive measurement techniques such as respiratory inductive plethysmography (RIP), electromagnetic inductance plethysmography (EIP) or IP.

Noninvasive measurement equipment. Some sort of a respiration-related measurement signal can be obtained with a variety of mechanical, acoustic, optical or electromagnetic instruments. However, if breath-to-breath tidal respiratory flow, instead of respiratory rate or other trivial measures, is tube analysed, the equipment selection is greatly narrower. Most TB studies with a clinical interest have used equipment that needs direct access at the airway opening, namely, a PNT with a face mask or mouth piece. PNT is considered the gold standard method and other less intrusive equipment are typically compared with PNT when their validity for tidal flow measurement is assessed. The most studied alternative for PNT is RIP. In RIP two elastic belts with a sewn-in electrical conductive coils are placed on rib cage and abdomen to account for upper and lower chest wall movement, respectively. The electrical inductance of each belt is proportional to their varying length and it is determined with electrical oscillation circuitry connected to each belt. RIP as well as IP enable measurements in mobile subjects, whereas another noninvasive option, EIP, only permits measurements at bedside with rather sizeable external instrumentation. There is evidence that with certain border conditions, RIP-derived chest wall movement signal may be linear with the lung volume signal. The comparison of mere Tptef/Te value, however, gives a somewhat limited picture on the linearity of the methods. There are also some studies on TB analysis using the noninvasive methods in infants as such without validation.

Breath averaging techniques. Individual breaths of TB are not considered representative of how the subject breaths. Instead, TB is recorded over a period of time and an averaged result is presented. There are two approaches to this: 1) Deriving TB parameters from each breath and presenting an average of these or 2) averaging the respiratory waveforms and presenting the TB parameters derived from the averaged waveform. The first approach is more straightforward and is more widely adopted in the clinical literature. Typically the mean of the TB parameter is presented. This approach is also endorsed by the ATS/ERS guideline. The second approach of waveform averaging is less trivial, but is potentially more beneficial with noisy respiratory signals. When averaging parameters, majority of the investigators have selected the cycles for the averaging manually. This has been done by an experienced operator by visually examining the recorded TB traces for sections of stable respiration. Computer selection of the cycles could, however, yield more reliable results. Their approach was in practice very close to taking the trimmed mean of Tptef/Te or other parameters from all breaths. Several ways of waveform averaging have been described. One example extracted the first four Fourier components of the flow signal from each breath. The amplitude and phase lag of each component of each breath was averaged and the flow waveform was reconstructed from the averaged amplitude and phase lag. The main shortcomings stem from the phase-time differences between individual breaths and the use of only four first frequency components that are not enough to describe the complex shape of a flow waveform. One proposed method where flow and volume waveforms are averaged with respect to their phase in the FVL. Its most apparent shortcoming is that it is not well defined if the phase angle does not increase monotonously. This may happen in FVLs with noisy recordings or when averaging for instance pressure-flow loops. One example presented a method in which the FVL is divided into segments of equal length. This method does not require for the loops to have monotonous phase angle information. They claim that the major pitfall of the method is that short noise impulses in the loop may spread over extended length.

Embodiments of the present invention may be implemented in software, hardware, application logic or a combination of software, hardware and application logic. In an example embodiment, the application logic, software or instruction set is maintained on any one of various conventional computer-readable media. In the context of this document, a “computer-readable medium” may be any media or means that can contain, store, communicate, propagate or transport the instructions for use by or in connection with an instruction execution system, apparatus, or device, such as a computer. A computer-readable medium may comprise a computer-readable storage medium that may be any media or means that can contain or store the instructions for use by or in connection with an instruction execution system, apparatus, or device, such as a computer. The exemplary embodiments can store information relating to various processes described herein. This information can be stored in one or more memories, such as a hard disk, optical disk, magneto-optical disk, RAM, and the like. One or more databases can store the information used to implement the exemplary embodiments of the present inventions. The databases can be organized using data structures (e.g., records, tables, arrays, fields, graphs, trees, lists, and the like) included in one or more memories or storage devices listed herein. The processes described with respect to the exemplary embodiments can include appropriate data structures for storing data collected and/or generated by the processes of the devices and subsystems of the exemplary embodiments in one or more databases.

All or a portion of the exemplary embodiments can be conveniently implemented using one or more general purpose processors, microprocessors, digital signal processors, micro-controllers, and the like, programmed according to the teachings of the exemplary embodiments of the present inventions, as will be appreciated by those skilled in the computer and/or software art(s). Appropriate software can be readily prepared by programmers of ordinary skill based on the teachings of the exemplary embodiments, as will be appreciated by those skilled in the software art. In addition, the exemplary embodiments can be implemented by the preparation of application-specific integrated circuits or by interconnecting an appropriate network of conventional component circuits, as will be appreciated by those skilled in the electrical art(s). Thus, the exemplary embodiments are not limited to any specific combination of hardware and/or software.

If desired, the different functions discussed herein may be performed in a different order and/or concurrently with each other.

Furthermore, if desired, one or more of the above-described functions may be optional or may be combined.

Although various aspects of the invention are set out in the independent claims, other aspects of the invention comprise other combinations of features from the described embodiments and/or the dependent claims with the features of the independent claims, and not solely the combinations explicitly set out in the claims.

It is obvious to a person skilled in the art that with the advancement of technology, the basic idea of the invention may be implemented in various ways. The invention and its embodiments are thus not limited to the examples described above; instead they may vary within the scope of the claims.

1. A method for measuring respiration using impedance pneumography over a duration of at least several minutes, several hours or over the duration of night sleep, the method comprising:
using at least one electrode configured to be in contact with an arm of a human body and at least one electrode configured to be in skin contact with the thorax of the human body;
defining impedance signal changes which relate to the respiratory volume changes or time-differentiated impedance signal changes which relate to the respiratory flow; and
analyzing variation over time in flow-time, volume-time, flow-volume curves, or derived numerical indices, or plain respiratory impedance signal or its time derivate over the duration of at least several minutes, several hours or over the duration of night sleep.

2. The method of claim 1, further comprising suppressing the cardiogenic part of the measured impedance signal.

3. The method of claim 1, further comprising deriving flow-time, volume-time, or flow-volume curves from individual or averaged multiple breaths from the impedance signal or the time-differentiated impedance signal.

4. The method of claim 1, wherein the numerical indices describe the flow-time, volume-time, or flow-volume curves.

5. The method of claim 4, wherein the indices relate to the shape of the curves, timing of peak values or other points of interest, or ratio of values or timings of two points of interest in the said curves.

6. The method of claim 1, further comprising analyzing the curves, indices, or signals regarding their variation over time using methods such as nonlinear dynamics, entropy, detrended fluctuation analysis, Lyapunov exponent, correlation dimension, recurrence plot, noise limit, frequency spectrum or using descriptive statistics such as mean, variance, or distribution analysis.

7. The method of claim 1, comprising using two electrodes configured to be in contact with one arm of the human body and two electrodes configured to be in skin contact with the thorax of the human body on the side opposite to the arm.

8. The method of claim 1, comprising using two electrodes configured to be in contact with opposite arms of the human body and two electrodes configured to be in skin contact with the thorax on opposite sides of the human body.

9. The method of claim 1, comprising preventing the skin contact between the arm and the torso by an insulation material configured to be positioned between the arm and the torso.

10. The method of claim 9, comprising the insulation material being configured to be a sleeve preventing the skin contact.

11. The method of claim 9, comprising the insulation material being configured to be a shirt or a vest preventing the skin contact.

12. An apparatus for measuring respiration using impedance pneumography over a duration of at least several minutes, several hours or over the duration of night sleep, the apparatus comprising:
using at least one electrode configured to be in contact with an arm of a human body and at least one electrode configured to be in skin contact with the thorax of the human body;
at least one processor and at least one memory including computer program code, the at least one memory and the computer program code arranged to, with the at least one processor, cause the apparatus at least to perform:
defining impedance signal changes which relate to the respiratory volume changes or time-differentiated impedance signal changes which relate to the respiratory flow; and
analyzing variation over time in flow-time, volume-time, flow-volume curves, or derived numerical indices, or plain respiratory impedance signal or its time derivate over the duration of at least several minutes, several hours or over the duration of night sleep.

13. The apparatus of claim 12, causing the apparatus at least to perform:
suppressing the cardiogenic part of the measured impedance signal.

14. The apparatus of claim 12, causing the apparatus at least to perform: deriving flow-time, volume-time, or flow-volume curves from individual or averaged multiple breaths from the impedance signal or the time-differentiated impedance signal.

15. The apparatus of claim 14, wherein the numerical indices describe the flow-time, volume-time, or flow-volume curves.

16. The apparatus of claim 15, wherein the indices relate to the shape of the curves, timing of peak values or other points of interest, or ratio of values or timings of two points of interest in the said curves.

17. The apparatus of claim 12, causing the apparatus at least to perform: analyzing the curves, indices, or signals regarding their variation over time using methods such as nonlinear dynamics, entropy, detrended fluctuation analysis, Lyapunov exponent, correlation dimension, recurrence plot, noise limit, frequency spectrum or using descriptive statistics such as mean, variance, or distribution analysis.

18. The apparatus of claim 12, comprising two electrodes configured to be in contact with one arm of the human body and two electrodes configured to be in skin contact with the thorax of the human body on the side opposite to the arm.

19. The apparatus of claim 12, comprising two electrodes configured to be in contact with opposite arms of the human body and two electrodes configured to be in skin contact with the thorax on opposite sides of the human body.

20. The apparatus of claim 12, comprising preventing the skin contact between the arm and the torso by an insulation material configured to be positioned between the arm and the torso.

21. The apparatus of claim 12, comprising the insulation material being configured to be a sleeve preventing the skin contact.

22. The apparatus of claim 12, comprising the insulation material being configured to be a shirt or a vest preventing the skin contact.
